(19) United States
(12) Patent Application Publication
(10) Pub. No.: US 2017/0247692 A1
(43) Pub. Date: Aug. 31, 2017

(54) COMPOSITIONS AND METHODS FOR TREATING NEUROLOGICAL DISORDERS

(71) Applicant: EFFECTOR THERAPEUTICS, INC., San Diego, CA (US)

(72) Inventors: James Appleman, San Diego, CA (US); Peggy A. Thompson, San Diego, CA (US); Vera Huang, San Diego, CA (US)

(21) Appl. No.: 15/114,277

(22) PCT Filed: Feb. 6, 2015
(86) PCT No.: PCT/US2015/014919

§ 371 Date: Jul. 26, 2016

Related U.S. Application Data
(60) Provisional application No. 61/937,315, filed on Feb. 7, 2014.

(51) Int. Cl. C12N 15/113; G01N 33/50

(52) U.S. Cl. CPC C12N 15/113; G01N 33/5023; G01N 33/5058; C12N 2310/14

(57) ABSTRACT

The present disclosure relates to compositions and methods for treating or preventing a neurological disorder.

### BACKGROUND

An exemplary neurodevelopmental disease is Fragile X syndrome, which is caused by a redundant trinucleotide (CGG) repeat in the 5′ UTR of the fragile X mental retardation 1 gene (FMR1). This causes silencing of the FMR1 gene at the transcriptional level and results in the lack of fragile X mental retardation 1 protein (FMRP) expression. FMRP is a cytoplasmic RNA binding protein that associates with polyribosomes as part of a large ribonucleoprotein complex and acts as a negative regulator of translation. Hence, FMRP is thought to regulate the translation of specific mRNAs that are critical for correct development of neurons and synaptic function. The Fragile X syndrome is directly linked to this lack of FMRP expression or loss of FMRP function (i.e., loss of translational control). Indeed, Fmr1 knockout mice have abnormal dendritic spines, which are thought to be the basis of the disease associated mental retardation (see, e.g., Darnell et al., Cell 146: 247, 2011).

There is a need in the art for new, effective methods of treating or preventing neurological disorders and for identifying biomarkers for use in developing therapeutic agents and assessing therapeutic response. The present disclosure meets such needs, and further provides other related advantages.

### BRIEF SUMMARY

In one aspect, the present disclosure provides a method for preventing, treating or ameliorating a neurological disorder, comprising administering to a subject having a neurological disorder a therapeutically effective amount of a modulator of any one of the genes listed in Table 2 or Table 3.

In another aspect, the present disclosure provides a method for reducing the risk of developing a neurological disorder, comprising: administering to a subject at risk of developing a neurological disorder a therapeutically effective amount of a modulator of any one of the genes listed in Table 2 or Table 3.

In certain embodiments, the neurological disorder being treated or for which the risk is being reduced is selected from Parkinson's disease, Amyotrophic Lateral Sclerosis (ALS), Creutzfeldt-Jakob disease, Huntington's disease, Lewy body dementia, frontotemporal dementia, corticobasal degeneration, primary progressive aphasia, progressive supranuclear palsy or Alzheimer's disease. In other embodiments, the neurological disorder is selected from autism, autism spectrum disorders, Fragile X Syndrome, attention deficit disorder, or pervasive development disorders.

### BRIEF DESCRIPTION OF THE DRAWINGS

FIG. 1 shows the translation levels of proteins associated with a neurodevelopmental disease model. Western blot analysis of the protein levels of FMRP, TSC2 and β-actin after siRNA knockdown of the FMR1 gene. SH-SY5Y cells were transfected with either siControl or siFMR1 at 100 nM for 3 days.

FIG. 2 shows the ribosomal profile of a neurodevelopmental disease model. Comparison of changes in mRNA levels (RNA) and translational rate (RPF) in SH-SY5Y neuronal cells transfected with either a control siRNA or test siFMR1. Data points in red have a p-value of <0.05 for changes in translational efficiency.

FIG. 3 shows the top up- and down-translationally regulated genes in a neurodevelopmental disease model. siRNA knockdown of the FMRP gene in SH-SY5Y cells with siFMR1 versus siControl. The top 20 up- or down-differentially translationally regulated genes show a 60 or 45%, respectively, enrichment for association with neurological disease and development (p-value≦0.05).

### DETAILED DESCRIPTION

The instant disclosure provides compositions and methods for identifying agents and validating targets for preventing, ameliorating or treating a neurological disorder or disease. For example, translational profiles may be used to (a) identify a candidate therapeutic against an neurological disorder-associated target for normalizing a translational profile associated with a neurological disorder, (b) validate a neurological disorder-associated target for normalizing a translational profile associated with a neurological disorder, or (c) identify a subject having or at risk of developing a neurological disorder as a candidate subject for treating or preventing the neurological disorder with a therapeutic agent against a neurological disorder-associated target.

By way of background, a neurological disorder is any disorder of involving the nervous system, such as structural, biochemical, electrical abnormalities, which may or may not have a genetic origin, in the brain, spinal cord, or other nerves that can have a range of symptoms. Neurological disorders can be categorized according to the primary location affected, the primary type of dysfunction involved, or the primary type of cause. For example, a neurological disorder may be neurodegenerative, neurocognitive, neurodevelopmental, or a combination thereof.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. The term “consisting essentially of” limits the scope of a claim to the specified materials or steps, or to those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have” and “comprise” are used synonymously, which terms and variants thereof are intended to be construed as non-limiting.

As used herein, the term “translational profile” refers to the amount of protein that is translated (i.e., translational level) for each gene in a given set of genes in a biological sample, collectively representing a set of individual translational rate values, translational efficiency values, or both translational rate and translational efficiency values for each of one or more genes in a given set of genes. In some embodiments, a translational profile comprises translational levels for a plurality of genes in a biological sample (e.g., cells), e.g., for at least about 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10,000 genes or more, or for at least about 1%, 2%, 3%, 4%, 5%, 6%, 7%, 8%, 9%, 10%, 15%, 20%, 25%, 50% or more of all genes in the sample. In some embodiments, a translational profile comprises a genome-wide measurement of translational rate, translational efficiency or both in a biological sample. In certain embodiments, a translational profile refers to a quantitative measure of the amount of mRNA associated with one or more ribosomes for each gene (i.e., translational rate, efficiency or both) in a given set of genes in a biological sample, wherein the amount of ribosome-associated mRNA correlates to the amount of protein that is translated (i.e., translational level).

As used herein, “translation rate” or “rate of translation” or “translational rate” refers to the total count of ribosome engagement, association or occupancy of mRNA for a particular gene as compared to the total count of ribosome engagement, association or occupancy of mRNA for at least one other gene or set of genes, wherein the count of total ribosomal occupancy correlates to the level of protein synthesis. Examination of translation rate across individual genes may be quantitative or qualitative, which will reveal differences in translation. In certain embodiments, translational rate provides a measure of protein synthesis for one or more genes, a plurality of genes, or across an entire genome. In particular embodiments, a translation rate is the amount of mRNA fragments protected by ribosomes for a particular gene relative to the amount of mRNA fragments protected by ribosomes for one or more other genes or groups of genes. For example, the mRNA fragments protected by ribosomes may correspond to a portion of the 5′-untranslated region, a portion of the coding region, a portion of a splice variant coding region, or combinations thereof. In further embodiments, the translation rate is a measure of one, a plurality or all mRNA variants of a particular gene. Translation rates can be established for one or more selected genes or groups of genes within a single composition (e.g., biological sample), between different compositions, or between a composition that has been split into at least two portions and each portion exposed to different conditions.

As used herein, “mRNA level” refers to the amount, abundance, or concentration of mRNA or portions thereof for a particular gene in a composition (e.g., biological sample). In certain embodiments, mRNA level refers to a count of one mRNA, a plurality of mRNA or all mRNA forms or fragments for a particular gene, including pre-mRNA, mature mRNA, or splice variants thereof. In particular embodiments, an mRNA level for one or more genes or groups of genes corresponds to counts of unique mRNA sequences or portions thereof for a particular gene that map to a 5′-untranslated region, a coding region, a splice variant coding region, or any combination thereof.

As used herein, “translation efficiency” or “translational efficiency” refers to the ratio of the translation rate for a particular gene to the mRNA level for a particular gene in a given set of genes. For example, gene X may produce an equal abundance of mRNA (i.e., same or similar mRNA level) in normal and diseased tissue, but the amount of protein X produced may be greater in diseased tissue as compared to normal tissue. In this situation, the message for gene X is more efficiently translated in diseased tissue than in normal tissue (i.e., an increased translation rate without an increase in mRNA level). In another example, gene Y may produce half the mRNA level in normal tissue as compared to diseased tissue, and the amount of protein Y produced in normal tissue is half the amount of protein Y produced in diseased tissue. In this second situation, the message for gene Y is translated equally efficiently in normal and diseased tissue (i.e., a change in translation rate in diseased tissue that is proportional to the increase in mRNA level and, therefore, the translational efficiency is unchanged). In other words, the expression of gene X is altered at the translational level, while gene Y is altered at the transcriptional level. In certain situations, both the amount of mRNA and protein may change such that mRNA abundance (transcription), translation rate, translation efficiency, or a combination thereof is altered relative to a particular reference or standard.

In certain embodiments, translational efficiency may be standardized by measuring a ratio of ribosome-associated mRNA read density (i.e., translation level) to mRNA abundance read density (i.e., transcription level) for a particular gene (see, e.g., Example 3). As used herein, “read density” is a measure of mRNA abundance and protein synthesis (e.g., ribosome profiling reads) for a particular gene, wherein at least 5, 10, 15, 20, 25, 50, 100, 150, 175, 200, 225, 250, 300 reads or more per unique mRNA or portion thereof is performed in relevant samples to obtain single-gene quantification for one or more treatment conditions. In certain embodiments, translational efficiency is scaled to standardize or normalize the translational efficiency of a median gene to 1.0 after excluding regulated genes (e.g., log₂ fold-change±1.5 after normalizing for the all-gene median), which corrects for differences in the absolute number of sequencing reads obtained for different libraries. In further embodiments, changes in protein synthesis, mRNA abundance and translational efficiency are similarly computed as the ratio of read densities between different samples and normalized to give a median gene a ratio of 1.0, normalized to the mean, normalized to the mean or median of log values, or the like.

As used herein, “gene signature” refers to a plurality of genes that exhibit a generally coherent, systematic, coordinated, unified, collective, congruent, or signature expression pattern or translation efficiency. In certain embodiments, a gene signature is (a) a plurality of genes that together comprise at least a detectable or identifiable portion of a biological pathway (e.g., 2, 3, 4, 5, or more genes; a neurological disorder-associated gene signature comprising, for example, up- or down-regulated genes from Table 2 or 3, respectively, or a combination thereof), (b) a complete set of genes associated with a biological pathway, or (c) a cluster or grouping of independent genes having a recognized pattern of expression (e.g., response to a known drug or active compound; related to a disease state such as a neurological disorder). One or more genes from a particular gene signature may be part of a different gene signature (e.g., a cell migration pathway may share a gene with a cell adhesion pathway)—that is, gene signatures may intersect or overlap but each signature can still be independently defined by its unique translation profile.

The term “modulate” or “modulator,” as used with reference to altering an activity of a target gene or signaling pathway, refers to increasing (e.g., activating, facilitating, enhancing, agonizing, sensitizing, potentiating, or up regulating) or decreasing (e.g., preventing, blocking, inactivating, delaying activation, desensitizing, antagonizing, attenuating, or down regulating) the activity of the target gene or signaling pathway. In certain embodiments, a modulator alters a translational profile at the translational level (i.e., increases or decreases translation rate, translation efficiency or both, as described herein), at the transcriptional level, or both.

In some embodiments, an agent that modulates translation in a neurological disorder is identified as suitable for use when one or more genes of one or more biological pathways, gene signatures or combinations thereof are differentially translated by at least 1.5-fold (e.g., at least 1.5-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold or more) in a first translational profile (e.g., treated neurological disorder sample or normal sample) as compared to a second translational profile (e.g., untreated neurological disorder sample). In some embodiments, an agent that modulates translation in a neurological disorder is identified as suitable for use when the translational rate, translational efficiency or both for one or more genes of one or more biological pathways, gene signatures or combinations thereof are increased or decreased by at least 1.5-fold (e.g., at least 1.5-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold or more) in a first translational profile as compared to a second translational profile.

A “biological sample” includes blood and blood fractions or products (e.g., serum, plasma, platelets, red blood cells, or the like); sputum or saliva; kidney, lung, liver, heart, brain, nervous tissue, thyroid, eye, skeletal muscle, cartilage, or bone tissue; cultured cells, e.g., primary cultures, explants, and transformed cells, stem cells, stool, urine, etc. Such biological samples (e.g., disease samples or normal samples) also include sections of tissues, such as a biopsy or autopsy sample, frozen sections taken for histologic purposes, or cells or other biological material used to model disease or to be representative of a pathogenic state (e.g., siFMR1 treated cells as a model system for Fragile X syndrome). In certain embodiments, a biological sample is obtained from a “subject,” e.g., a eukaryotic organism, most preferably a mammal such as a primate, e.g., chimpanzee or human; cow; dog; cat; rodent, e.g., guinea pig, rat, or mouse; rabbit; bird; reptile; or fish.

As used herein, the term “normalize” or “normalizing” or “normalization” refers to adjusting the translational rate, translational efficiency, or both of one or more genes in a biological sample from a subject (e.g., a disease sample from one or more subjects, tissues or organs) to a level that is more similar, closer to, or comparable to the translational rate, translational efficiency, or both of those same one or more genes in a control sample (e.g., a non-diseased or normal sample from the same or different subject, tissue or organ). In certain embodiments, normalization refers to modulation of one or more translational regulators or translational system components to adjust or shift the translational rate, efficiency or both of one or more genes in a biological sample (e.g., diseased, abnormal or other biologically altered condition) to a translational efficiency that is more similar, closer to or comparable to the translational efficiency of those one or more genes in a non-diseased or normal control sample. In some embodiments, normalization is evaluated by determining a translational rate, translational efficiency or both of one or more genes in a biological sample (e.g., disease sample) from a subject before and after an agent (e.g., therapeutic or known active agent) is administered to the subject and comparing the translational rate, translational efficiency or both before and after administration to the translational rate, translational efficiency or both from a control sample in the absence or presence of the agent.

As used herein, the phrase “differentially translated” refers to a change or difference (e.g., increase, decrease or a combination thereof) in translation rate, translation efficiency, or both of one gene, a plurality of genes, a set of genes of interest, one or more gene clusters, or one or more gene signatures under a particular condition as compared to the translation rate, translation efficiency, or both of the same gene, plurality of genes, set of genes of interest, gene clusters, or gene signatures under a different condition, which is observed as a difference in expression pattern. For example, a translational profile of a diseased cell may reveal that one or more genes have higher translation rates, higher translation efficiencies, or both (e.g., higher ribosome engagement of mRNA or higher protein abundance) than observed in a normal cell. In some embodiments, one or more gene signatures, gene clusters or sets of genes of interest are differentially translated in a first translational profile as compared to one or more other translational profiles. In further embodiments, one or more genes, gene signatures, gene clusters or sets of genes of interest in a first translational profile show at least a 1.5-fold translation differential or at least a 1.0 log₂ change (i.e., increase or decrease) as compared to the same one or more genes in at least one other different (e.g., second, third, etc.) translational profile.

In some embodiments, two or more translational profiles are generated and compared to each other to determine the differences (i.e., increases and/or decreases in translational rate, translational efficiency, or both) for each gene in a given set of genes between the two or more translational profiles. The comparison between the two or more translational profiles is referred to as the “differential translational profile.” In certain embodiments, a differential translational profile comprises one or more genes, gen clusters, or gene signatures (e.g., a neurological disorder-associated pathway), or combinations thereof.

In certain embodiments, differential translation between genes or translational profiles may involve or result in a biological (e.g., phenotypic, physiological, clinical, therapeutic, prophylactic) benefit. For example, when identifying a therapeutic, validating a target, or treating a subject having a neurological disorder or disease, a “biological benefit” means that the effect on translation rate, translation efficiency or both, or the effect on the translation rate, translation efficiency or both of one or more genes of a translational profile allows for intervention or management of the neurological disorder or disease of a subject (e.g., a human or non-human mammal, such as a primate, horse, dog, mouse, rat). In general, one or more differential translations or differential translation profiles indicate that a “biological benefit” will be in the form, for example, of an improved clinical outcome; lessening or alleviation of symptoms associated with neurological disorder; decreased occurrence of symptoms; improved quality of life; longer disease-free status; diminishment of extent of neurological disorder; stabilization of a neurological disorder; delay of neurological disorder progression; remission; survival; or prolonging survival. In certain embodiments, a biological benefit comprises normalization of a differential translation profile, or comprises a shift in translational profile to one closer to or comparable to a translational profile induced by a known active compound or therapeutic, or comprises inducing, stimulating or promoting a desired phenotype or outcome (e.g., phenotypic reversal, quiescence, cellular repair, apoptosis, necrosis, cytotoxicity), or reducing, inhibiting or preventing an undesired phenotype or outcome (e.g., transformation, proliferation, migration).

In some embodiments, less than about 20% of the genes in the genome are differentially translated by at least 1.5-fold in a first translational profile as compared to a second translational profile. In some embodiments, less than about 5% of the genes in the genome are differentially translated by at least 2-fold or at least 3-fold in a first translational profile as compared to a second translational profile. In some embodiments, less than about 1% of the genes in the genome are differentially translated by at least 4-fold or at least 5-fold in a first translational profile as compared to a second translational profile.

As described herein, differentially translated genes between first and second translational profiles under a first condition may exhibit translational profiles “closer to” each other (i.e., identified through a series of pair-wise comparisons to confirm a similarity of pattern) under one or more different conditions (e.g., differentially translated genes between a normal sample and a neurological disorder sample may have a more similar translational profile when the normal sample is compared to a neurological disorder sample contacted with a candidate agent; differentially translated genes between a neurological disorder sample and a neurological disorder sample treated with a known active agent may have a more similar translational profile when the disease sample treated with a known active agent is compared to the disease sample contacted with a candidate agent). In certain embodiments, a test translational profile is “closer to” a reference translational profile when at least 99%, 95%, 90%, 80%, 70%, 60%, 50%, 25%, or 10% of a selected portion of differentially translated genes, a majority of differentially translated genes, or all differentially translated genes show a translational profile within 75%, 70%, 65%, 60%, 55%, 50%, 45%, 40%, 35%, 30%, or 25%, respectively, of their corresponding genes in the reference translational profile. In further embodiments, a selected portion of differentially translated genes, a majority of differentially translated genes, or all differentially translated genes from an experimental translational profile have a translational profile “closer to” the translational profile of the same genes in a reference translational profile when the amount of protein translated in the experimental and reference translational profiles are within about 3.0 log₂, 2.5 log₂, 2.0 log₂, 1.5 log₂, 1.1 log₂, 0.5 log₂, 0.2 log₂ or closer. In still further embodiments, a selected portion of differentially translated genes, a majority of differentially translated genes, or all differentially translated genes from an experimental translational profile have a translational profile “closer to” the translational profile of the same genes in a reference translational profile when the amount of protein translated in the experimental and reference translational profiles differs by no more than about 50%, 45%, 40%, 35%, 30%, 25%, 20%, 15%, 10%, 5%, 1% or less.

In some embodiments, an experimental differential profile as compared to a reference differential translational profile of interest has at least a 1.0 log₂ change in translational rate, translational efficiency, or both for at least 0.05%, at least 0.1%, at least 0.25%, at least 0.5%, at least 1%, at least 2%, at least 3%, at least 4%, at least 5%, at least 6%, at least 7%, at least 8%, at least 9%, at least 10%, at least 15%, at least 20%, at least 30%, at least 40%, at least 50%, at least 60%, at least 70%, at least 80%, or at least 90% or more of a set of selected differentially translated genes or for the entire set of selected differentially translated genes. In some embodiments, an experimental differential profile as compared to a reference differential translational profile of interest has at least a 2 log₂ change in translational rate, translational efficiency, or both for at least 0.05%, at least 0.1%, at least 0.25%, at least 0.5%, at least 1%, at least 5%, at least 10%, at least 15%, at least 20%, at least 30%, at least 40%, at least 50%, at least 60%, at least 70%, at least 80%, or at least 90% or more of a set of selected differentially translated genes or for the entire set of differentially translated genes. In some embodiments, an experimental differential profile as compared to a reference differential translational profile of interest has at least a 3 log₂ change in translational rate, translational efficiency, or both for at least 0.05%, at least 0.1%, at least 0.25%, at least 0.5%, at least 1%, at least 5%, at least 10%, at least 15%, at least 20%, at least 30%, at least 40%, at least 50%, at least 60%, at least 70%, at least 80%, or at least 90% or more of a set of selected differentially translated genes or for the entire set of selected differentially translated genes. In some embodiments, an experimental differential profile as compared to a reference differential translational profile of interest has at least a 4 log₂ change in translational levels for at least 0.05%, at least 0.1%, at least 0.25%, at least 0.5%, at least 1%, at least 5%, at least 10%, at least 15%, at least 20%, at least 30%, at least 40%, at least 50%, at least 60%, at least 70%, at least 80%, or at least 90% or more of a set of selected differentially translated genes or for the entire set of selected differentially translated genes.

As described herein, a differential translational profile between a first sample and a control may be “comparable” to a differential translational profile between a second sample and the control (e.g., the differential profile between a neurological disorder sample and the neurological disorder sample treated with a known active compound may be comparable to the differential profile between the neurological disorder sample and the neurological disorder sample contacted with a candidate agent; the differential profile between a neurological disorder sample and a non-diseased (normal) sample may be comparable to the differential profile between the neurological disorder sample and the neurological disorder sample contacted with a candidate agent). In certain embodiments, a test differential translational profile is “comparable to” a reference differential translational profile when at least 99%, 95%, 90%, 80%, 70%, 60%, 50%, 25%, or 10% of a selected portion of differentially translated genes, a majority of differentially translated genes, or all differentially translated genes show a translational profile within 75%, 70%, 65%, 60%, 55%, 50%, 45%, 40%, 35%, 30%, or 25%, respectively, of their corresponding genes in the reference translational profile. In further embodiments, a differential translational profile comprising a selected portion of the differentially translated genes or all the differentially translated genes has a differential translational profile “comparable to” the differential translational profile of the same genes in a reference differential translational profile when the amount of protein translated in the experimental and reference differential translational profiles are within about 3.0 log₂, 2.5 log₂, 2.0 log₂, 1.5 log₂, 1.0 log₂, 0.5 log₂, 0.2 log₂ or closer. In still further embodiments, a differential translational profile comprising a selected portion of the differentially translated genes or all the differentially translated genes has a differential translational profile “comparable to” the differential translational profile of the same genes in a reference differential translational profile when the amount of protein translated in the experimental and reference differential translational profiles differs by no more than about 50%, 45%, 40%, 35%, 30%, 25%, 20%, 15%, 10%, 5%, 1% or less.

The term “neurological disorder” or “neurological disease” refers to any medical condition resulting in a disturbance of normal functioning of any portion of the central or peripheral nervous system, including the brain, spine, or other nerves. Exemplary neurological disorders include epilepsy, Alzheimer's disease and other dementias, cerebrovascular diseases including stroke, migraine and other headache disorders, multiple sclerosis, Parkinson's disease, neuroinfections, brain tumors, traumatic disorders of the nervous system such as brain trauma, and neurological disorders as a result of malnutrition. A neurological disorder or disease may be categorized as neurodegenerative, neurocognitive, neurodevelopmental, neurobehavioral, or the like. In some embodiments, a neurological disease is a neurodegenerative disease (e.g., Parkinson's disease, Amyotrophic Lateral Sclerosis (ALS), Creutzfeldt-Jakob disease, Huntington's disease, Lewy body dementia, frontotemporal dementia, corticobasal degeneration, primary progressive aphasia, progressive supranuclear palsy or Alzheimer's disease). In some embodiments, a neurological disease is a neurocognitive or neurodevelopmental disease (e.g., autism, autism spectrum disorders, Fragile X Syndrome, attention deficit disorder, pervasive development disorders).

“Treatment,” “treating” or “ameliorating” refers to medical management of a disease, disorder, or condition of a subject (i.e., patient), which may be therapeutic, prophylactic/preventative, or a combination treatment thereof. A treatment may improve or decrease the severity at least one symptom of neurological disorder, delay worsening or progression of a disease, or delay or prevent onset of additional associated diseases. “Reducing the risk of developing a neurological disorder” refers to preventing or delaying onset of a neurological disorder or reoccurrence of one or more symptoms of the neurological disorder.

A “therapeutically effective amount (or dose)” or “effective amount (or dose)” of a compound refers to that amount sufficient to result in amelioration of one or more symptoms of the disease being treated in a statistically significant manner. When referring to an individual active ingredient administered alone, a therapeutically effective dose refers to that ingredient alone. When referring to a combination, a therapeutically effective dose refers to combined amounts of the active ingredients that result in the therapeutic effect, whether administered serially or simultaneously.

The term “pharmaceutically acceptable” refers to molecular entities and compositions that do not produce allergic or other serious adverse reactions when administered to a subject using routes well-known in the art.

A “subject in need” refers to a subject at risk of, or suffering from, a disease, disorder or condition that is amenable to treatment or amelioration with a compound or a composition thereof provided herein. In certain embodiments, a subject in need is a human.

The “percent identity” between two or more nucleic acid sequences is a function of the number of identical positions shared by the sequences (i.e., % identity=number of identical positions/total number of positions×100), taking into account the number of gaps, and the length of each gap that needs to be introduced to optimize alignment of two or more sequences. The comparison of sequences and determination of percent identity between two or more sequences can be accomplished using a mathematical algorithm, such as BLAST and Gapped BLAST programs at their default parameters (e.g., Altschul et al., J. Mol. Biol. 215:403, 1990; see also BLASTN at www.ncbi.nlm.nih.gov/BLAST).

A “conservative substitution” is recognized in the art as a substitution of one amino acid for another amino acid that has similar properties. Exemplary conservative substitutions are well known in the art (see, e.g., WO 97/09433, p. 10; Lehninger, Biochemistry, 2^{nd} Edition; Worth Publishers, Inc. NY:NY (1975), pp. 71-77; Lewin, Genes IV, Oxford University Press, NY and Cell Press, Cambridge, Mass. (1990), p. 8).

### Neurological Disorder or Disease

In one aspect, the present disclosure provides a method for preventing, treating or ameliorating a neurological disorder, comprising administering to a subject having a neurological disorder a therapeutically effective amount of a modulator of any one or more of the genes (including any alleles, homologs, or orthologs) listed in Tables 1-3 or encoded products thereof (including any active fragments or splice variants thereof). In certain embodiments, the present disclosure provides a method for reducing the risk of developing a neurological disorder, comprising administering to a subject at risk of developing a neurological disorder a therapeutically effective amount of a modulator of any one or more of the genes (including any alleles, homologs, or orthologs) listed in Tables 1-3 or encoded products thereof (including any active fragments or splice variants thereof).

In some embodiments, the present disclosure provides a method for treating a neurological disorder, comprising administering to a subject having a neurological disorder a therapeutically effective amount of a modulator specific for any one or more of the genes (including any alleles, homologs, or orthologs) listed in Tables 1-3 or encoded products (including any active fragments or splice variants thereof). In other embodiments, the present disclosure provides a method for reducing the risk of developing a neurological disorder, comprising administering to a subject at risk of developing a neurological disorder a therapeutically effective amount of a modulator specific for any one or more of the genes (including any alleles, homologs, or orthologs) listed in Tables 1-3 or encoded products thereof (including any active fragments or splice variants thereof).

As used herein, a modulator or agent that “specifically binds” or is “specific for” a target refers to an association or union of a modulator or agent (e.g., siRNA, chemical compound) to a target molecule (e.g., a nucleic acid molecule encoding a target, a target product encoded by a nucleic acid molecule, or a target activity), which may be a covalent or non-covalent association, while not significantly associating or uniting with any other molecules or components in a cell, tissue, biological sample, or subject. For example, a specific modulator may be an siRNA or a derivative thereof (e.g., nuclease resistant modifications, such as phosphorothioate, locked nucleic acids (LNA), 2′-O-methyl modifications, morpholino linkages, or the like).

In another aspect, the instant disclosure provides a method for identifying a candidate therapeutic for normalizing a translational profile associated with a neurological disorder, comprising (a) determining three independent translational profiles, each for a plurality of genes, wherein (i) a first translational profile is from a neurological disorder sample, (ii) a second translational profile is from (1) a control non-diseased sample or (2) a control non-diseased sample contacted with a candidate agent, and (iii) a third translational profile is from the neurological disorder sample contacted with a candidate agent; (b) determining a first differential translational profile comprising one or more genes differentially translated in the first translational profile as compared to the second translational profile, and determining a second differential translational profile comprising one or more genes differentially translated in the first translational profile as compared to the third translational profile, wherein the one or more differentially translated genes are selected from the genes listed in Tables 1-3; and (c) identifying the agent as a candidate therapeutic for normalizing a translational profile associated with the neurological disorder when the first differential translational profile is comparable to the second differential translational profile.

In still another aspect, the instant disclosure provides a method for validating a target for normalizing a translational profile associated with a neurological disorder, the method comprising (a) determining three independent translational profiles, each for a plurality of genes, wherein (i) a first translational profile is from a neurological disorder, a neurodegenerative disease, a neurodevelopmental disease, a metabolic disease, or a viral infection sample, (ii) a second translational profile is from (1) a control non-diseased sample or (2) a control non-diseased sample contacted with an agent that modulates a target, and (iii) a third translational profile is from the neurological disorder sample contacted with the agent that modulates the target; (b) determining a first differential translational profile comprising one or more genes differentially translated in the first translational profile as compared to the second translational profile, and determining a second differential translational profile comprising one or more genes differentially translated in the first translational profile as compared to the third translational profile, wherein the one or more differentially translated genes are selected from the genes listed in Tables 1-3; and (c) validating the target as a target for normalizing a translational profile associated with the neurological disorder when the first differential translational profile is comparable to the second differential translational profile.

In some aspects, the instant disclosure provides a method of identifying a subject as a candidate for preventing, treating or ameliorating a neurological disorder with a therapeutic agent, the method comprising (a) determining a first translational profile for a plurality of genes in a sample from a subject having or suspected of having a neurological disorder; (b) determining a second translational profile for a plurality of genes in a control sample, wherein the control sample is from a subject known to respond to the therapeutic agent and wherein the sample has not been contacted with the therapeutic agent; and (c) identifying the subject as a candidate for treating neurological disorder with the therapeutic agent when the translational profile for one or more genes selected from Tables 1-3 of the first translational profile are comparable to the translational profile of the corresponding genes in the second translational profile. In a related aspect, the instant disclosure provides a method for preventing, treating or ameliorating a neurological disorder, comprising administering a therapeutic agent to a subject identified according to the method of identifying a subject as a candidate for preventing, treating or ameliorating a neurological disorder, thereby treating the subject.

In any of the aforementioned embodiments, the neurological disorder or disease may be Parkinson's disease, Amyotrophic Lateral Sclerosis (ALS), Creutzfeldt-Jakob disease, Huntington's disease, Lewy body dementia, frontotemporal dementia, corticobasal degeneration, primary progressive aphasia, progressive supranuclear palsy or Alzheimer's disease. In some embodiments, a neurological disorder or disease is autism, autism spectrum disorders, Fragile X Syndrome, attention deficit disorder, pervasive development disorders. In further embodiments, a modulator is formulated with a pharmaceutically acceptable diluent, carrier or excipient. In still further embodiments, a modulator is administered in combination with a second therapeutic agent. In any of the aforementioned embodiments, the subject is a human.

Subjects in need of administration of therapeutic agents as described herein include subjects at high risk for developing a neurological disorder as well as subjects presenting with an existing neurological disorder. A subject may be at high risk for developing a neurological disorder if the subject has been experienced an injury (e.g., exposure to certain medications or infectious agents) or has certain genetic mutations, or the like. Subjects suffering from or suspected of having a neurological disorder can be identified using methods as described herein. A subject may be any organism capable of developing a neurological disorder, such as humans, pets, livestock, show animals, zoo specimens, or other animals. For example, a subject may be a human, a non-human primate, dog, cat, rabbit, horse, or the like.

The therapeutic agents or pharmaceutical compositions that treat or reduce the risk of developing a neurological disorder provided herein are administered to a subject who has or is at risk of developing a neurological disorder at a therapeutically effective amount or dose. Such a dose may be determined or adjusted depending on various factors including the specific therapeutic agents or pharmaceutical compositions, the routes of administration, the subject's condition, that is, stage of the disease, severity of symptoms caused by the disease, general health status, as well as age, gender, and weight, and other factors apparent to a person skilled in the medical art. Similarly, the dose of the therapeutic for treating a disease or disorder may be determined according to parameters understood by a person skilled in the medical art. When referring to a combination, a therapeutically effective dose refers to combined amounts of the active ingredients that result in the therapeutic effect, whether administered serially or simultaneously (in the same formulation or concurrently in separate formulations). Optimal doses may generally be determined using experimental models and/or clinical trials. Design and execution of pre-clinical and clinical studies for a therapeutic agent (including when administered for prophylactic benefit) described herein are well within the skill of a person skilled in the relevant art.

Generally, the therapeutic agent is administered at a therapeutically effective amount or dose. A therapeutically effective amount or dose will vary according to several factors, including the chosen route of administration, formulation of the composition, patient response, severity of the condition, the subject's weight, and the judgment of the prescribing physician. The dosage can be increased or decreased over time, as required by an individual patient. In certain instances, a patient initially is given a low dose, which is then increased to an efficacious dosage tolerable to the patient. Determination of an effective amount is well within the capability of those skilled in the art.

The route of administration of a therapeutic agent can be oral, intraperitoneal, transdermal, subcutaneous, by intravenous or intramuscular injection, by inhalation, topical, intralesional, infusion; liposome-mediated delivery; topical, intrathecal, gingival pocket, rectal, intrabronchial, nasal, transmucosal, intestinal, ocular or otic delivery, or any other methods known in the art.

In some embodiments, a therapeutic agent is formulated as a pharmaceutical composition. In some embodiments, a pharmaceutical composition incorporates particulate forms, protective coatings, protease inhibitors, or permeation enhancers for various routes of administration, including parenteral, pulmonary, nasal and oral. The pharmaceutical compositions can be administered in a variety of unit dosage forms depending upon the method/mode of administration. Suitable unit dosage forms, including powders, tablets, pills, capsules, lozenges, suppositories, patches, nasal sprays, injectables, implantable sustained-release formulations, etc.

In some embodiments, a pharmaceutical composition comprises an acceptable diluent, carrier or excipient. A pharmaceutically acceptable carrier includes any solvent, dispersion media, or coating that are physiologically compatible and that preferably do not interfere with or otherwise inhibit the activity of the therapeutic agent. Preferably, a carrier is suitable for intravenous, intramuscular, oral, intraperitoneal, transdermal, topical, or subcutaneous administration. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers. Other pharmaceutically acceptable carriers and their formulations are well-known and generally described in, for example, Remington: The Science and Practice of Pharmacy, 21st Edition, Philadelphia, Pa. Lippincott Williams & Wilkins, 2005. Various pharmaceutically acceptable excipients are well-known in the art and can be found in, for example, Handbook of Pharmaceutical Excipients (5^{th} ed., Ed. Rowe et al., Pharmaceutical Press, Washington, D.C.).

### EXAMPLES

### Example 1

### Effect of FMR1 Knockdown on Neurodevelopmental Disease

Fragile X syndrome is caused by a redundant trinucleotide (CGG) repeat in the 5′ UTR of the fragile X mental retardation 1 gene (FMR1), which silences the FMR1 gene at the transcriptional level and results in the lack of fragile X mental retardation 1 protein (FMRP) expression. FMRP is a cytoplasmic RNA binding protein that associates with polyribosomes as part of a large ribonucleoprotein complex and acts as a negative regulator of translation. Hence, FMRP is thought to regulate the translation of specific mRNAs that are critical for correct development of neurons and synaptic function. The Fragile X syndrome is directly linked to this lack of FMRP expression or loss of FMRP function (i.e., loss of translational control). Accordingly, an FMR1 knockdown assay was used as a model to examine neurodevelopmental disease.

Briefly, human neuroblastoma cells (SH-SY5Y, ATCC, passage 8) were transfected using Lipofectamine RNAiMax (Invitrogen) according to manufacturer's protocol with either siControl (Invitrogen, AM4611) or siFMR1 (Invitrogen) at 100 nM and cultured for 3 days in a humidified atmosphere of 5% CO₂ maintained at 37° C. in F12/DMEM media (1:1 ratio) supplemented with penicillin G (100 U/ml), streptomycin (100 μg/ml), and 10% FBS.

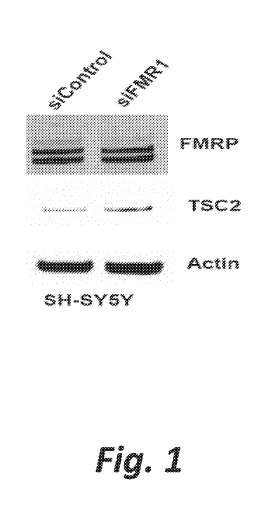

Protein levels of FMRP and TSC2 (a known translational target of FMRP) were evaluated by western blot analysis, and β-actin was used as a loading control (FIG. 1). Briefly, about 5×10⁵ transfected cells/well of a 6-well plate were harvested, washed with PBS, and lysed in 1× cell lysis buffer (Cell Signaling) for 15 min. at 4° C. Lysates were briefly sonicated, clarified by centrifugation for 15 min. at 14,000 rpm, and then supernatants were collected. Protein concentration in the soluble fraction was determined by BCA protein assay (Thermo Scientific). Samples of protein (20 μg) were resolved on 4-20% Bis-Tris gradient gel (Invitrogen) and transferred to nitrocellulose membrane. The resulting blots were blocked for 1 hr. at room temperature with Odyssey blocking solution (LI-COR) and then incubated with primary antibodies at 4° C. overnight. The following day, each blot was washed three times for 10 min. in TBST, and then incubated with IR-conjugated anti-rabbit IgG and anti-mouse IgG secondary antibody (IRDye 800 CW at 1:20,000; LI-COR) for 1 hr. at room temperature. The blots were washed and scanned, and then specific proteins were detected by using the LI-COR Odyssey infrared imager. The following antibodies (Cell Signaling) were used at 1:1000 dilution: anti-FMRP (#4317), anti-TSC2 (#4308), and anti-β-actin (#4970).

### Results

The uppermost band observed in the western blot analysis represents the FMR1 isoform and is sensitive to the siFMR1 knockdown. An approximately 30% knockdown efficiency of FMRP was determined by integrating the band intensities, as well as quantitating by q-PCR analysis (data not shown). The protein expression levels of TSC2 increased after knocking down FMRP, a negative translational regulator.

### Conclusion

Administration of siRNA specific for FMR1 mRNA shows that the protein levels of FMRP and TSC2 are altered and provide a model for examining Fragile X syndrome.

### Example 2

### Translational Profile of Neurodevelopmental Disease

Ribosomal profiling allows for measurement of changes in transcription and translation on a genome-wide basis accompanying siFMR1-induced Fragile X syndrome phenotype in human neuroblastoma cells. Ribosomal profiles of the siFMR1-treated SH-SY5Y cells from Example 1 (about 3×10⁶ cells/10 cm plate were harvested for ribosome profiling following siRNA transfection) were prepared and analyzed for changes in translational efficiencies with respect to potential disease-associated cellular changes accompanying this siFMR1-induced Fragile X syndrome phenotype.

Briefly, cells were washed with cold PBS supplemented with cycloheximide and lysed with 1× mammalian cell lysis buffer for 10 min. on ice. Lysates were clarified by centrifugation for 10 min. at 14,000 rpm and supernatants were collected. Cell lysates were processed to generate ribosomal protected fragments and total mRNA according to the instructions included with the ARTseq Ribosome Profiling Kit (Illumina). Sequencing of total RNA (RNA) and of ribosome-protected fragments of RNA (RPF) was carried out using RNA-Seq methodology according to the manufacturer's instructions (Illumina). To analyze the ribosomal profiles, RNA-Seq reads were processed with tools from the FASTX-Toolkit (fastq_quality trimmer, fastx clipper and fastx trimmer). Unprocessed and processed reads were evaluated for a variety of quality measures using FastQC. Processed reads were mapped to the human genome using Tophat. Gene-by-gene assessment of the number of fragments strictly and uniquely mapping to the coding region of each gene was conducted using HTSeq-count, a component of the HTSeq package. Differential analyses of the knockdown of the FMR1 gene were carried out with the software packages DESeq for transcription (RNA counts) and translational rate (RPF counts) and BABEL for translational efficiency based upon ribosomal occupancy as a function of RNA level (RNA and RPF counts). Genes with low counts in either RPF or RNA were excluded from differential analyses.

### Results

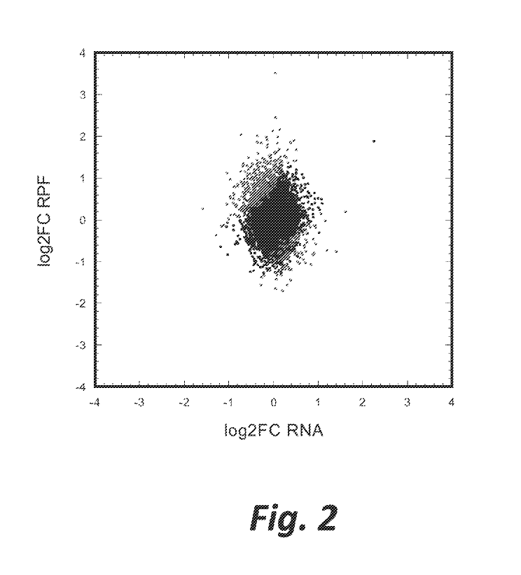

Ribosomal profiling was used to measure changes in transcription and translation on a genome-wide basis after transfecting the cells with either siControl or siFMR1. Analysis of the sequencing results for the FMR1 gene shows that about 30% reduction was observed, consistent with the western blot and q-PCR analyses. The FMRP specific target, TSC2, showed a corresponding approximate 30% increase in the translational rate in the absence of a change in transcriptional levels. On a genome-wide evaluation, knockdown of the FMR1 gene resulted in minimal changes in the transcriptome (see FIG. 2) with only a log₂ fold change of 2.3 and 1.6 for the top two up-regulated genes (log₂ fold change of −1.6 and -1.2 for the top two down-regulated genes). Changes in the translational rate were identified for a number of genes in the absence of a change in transcriptional levels, corresponding to a change in the translational efficiency (see Tables 1-3). These results indicate that FMRP is responsible for the translational regulation of this specific set of genes.

TABLE 1
Gene Signature for Neurodevelopmental Disease

| | | TE log₂Fold | BABEL | BABEL |
|---|---|---|---|---|
| Gene | HGNC | Change | p-value | FDR |
| ENSG00000178531 | CTXN1 | 3.47334116 | 2.08E−12 | 1.94E-08 |
| ENSG00000167378 | IRGQ | 2.768919604 | 1.29E−11 | 6.03E-08 |
| ENSG00000172936 | MYD88 | 2.167264278 | 5.48E−08 | 0.000171 |
| ENSG00000185043 | CIB1 | −2.157304408 | 6.63E−07 | 0.001552 |
| ENSG00000218891 | ZNF579 | 1.929654551 | 1.19E−06 | 0.002231 |
| ENSG00000243449 | C4orf48 | 1.805476999 | 1.68E−06 | 0.002492 |
| ENSG00000230055 | CISD3 | 2.022401614 | 1.86E−06 | 0.002492 |
| ENSG00000104859 | CLASRP | 1.640194908 | 6.54E−06 | 0.007013 |
| ENSG00000077463 | SIRT6 | 1.664140783 | 6.74E−06 | 0.007013 |
| ENSG00000161179 | YDJC | 1.877907938 | 1.06E−05 | 0.009906 |
| ENSG00000127540 | UQCR11 | 1.530529479 | 3.05E−05 | 0.02596 |
| ENSG00000147804 | SLC39A4 | 1.981071008 | 5.21E−05 | 0.038152 |
| ENSG00000164086 | DUSP7 | 1.764926734 | 5.30E−05 | 0.038152 |
| ENSG00000223496 | EXOSC6 | 1.868982284 | 6.39E−05 | 0.042715 |
| ENSG00000185386 | MAPK11 | 2.372609311 | 6.85E−05 | 0.042729 |
| ENSG00000173918 | C1QTNF1 | 1.92537716 | 7.82E−05 | 0.045747 |
| ENSG00000023909 | GCLM | −1.730520111 | 9.15E−05 | 0.04698 |
| ENSG00000112208 | BAG2 | −1.701624924 | 9.16E−05 | 0.04698 |
| ENSG00000101298 | SNPH | −1.917212168 | 9.54E−05 | 0.04698 |
| ENSG00000170873 | MTSS1 | −1.70546699 | 0.000103 | 0.048358 |
| ENSG00000172070 | SRXN1 | 1.796290905 | 0.00011 | 0.048838 |
| ENSG00000103121 | C16orf61 | −1.903559838 | 0.000115 | 0.048838 |
| ENSG00000135414 | GDF11 | 2.140837902 | 0.000128 | 0.052142 |
| ENSG00000125611 | CHCHD5 | 1.89475633 | 0.000142 | 0.055281 |
| ENSG00000078967 | UBE2D4 | 2.397910749 | 0.000204 | 0.075555 |
| ENSG00000099822 | HCN2 | 1.565382187 | 0.000213 | 0.075555 |
| ENSG00000165704 | HPRT1 | −1.342259199 | 0.000218 | 0.075555 |
| ENSG00000107872 | FBXL15 | 1.565215051 | 0.00027 | 0.087286 |
| ENSG00000198816 | ZNF358 | 2.10765993 | 0.000272 | 0.087286 |
| ENSG00000167685 | ZNF444 | 1.48065118 | 0.00028 | 0.087286 |
| ENSG00000167619 | TMEM145 | 1.629208724 | 0.000295 | 0.087661 |
| ENSG00000162522 | KIAA1522 | 1.447471049 | 0.0003 | 0.087661 |
| ENSG00000106635 | BCL7B | 2.054607285 | 0.00036 | 0.102215 |
| ENSG00000162585 | C1orf86 | 1.356164732 | 0.000376 | 0.103385 |
| ENSG00000167987 | VPS37C | 1.647488 | 0.000431 | 0.111143 |
| ENSG00000241839 | PLEKHO2 | 1.61326718 | 0.000435 | 0.111143 |
| ENSG00000115561 | CHMP3 | 2.222179181 | 0.000439 | 0.111143 |
| ENSG00000130522 | JUND | 1.805678938 | 0.000485 | 0.11938 |
| ENSG00000130529 | TRPM4 | 1.600245518 | 0.000528 | 0.122891 |
| ENSG00000158863 | FAM160B2 | 1.554486573 | 0.000531 | 0.122891 |
| ENSG00000168993 | CPLX1 | 1.524631252 | 0.000562 | 0.122891 |
| ENSG00000198947 | DMD | 1.882044683 | 0.000563 | 0.122891 |
| ENSG00000049769 | PPP1R3F | 1.895174442 | 0.000571 | 0.122891 |
| ENSG00000171159 | C9orf16 | 1.666128258 | 0.000578 | 0.122891 |
| ENSG00000100167 | MARCH11 | 1.673960211 | 0.000614 | 0.127726 |
| ENSG00000196072 | BLOC1S2 | 2.155264766 | 0.000642 | 0.130577 |
| ENSG00000101439 | CST3 | 1.312474345 | 0.000723 | 0.143914 |
| ENSG00000162981 | FAM84A | 1.511537171 | 0.000747 | 0.145677 |
| ENSG00000223802 | CERS1 | 1.711509695 | 0.000795 | 0.148774 |
| ENSG00000141933 | TPGS1 | 1.652396801 | 0.000811 | 0.148774 |
| ENSG00000162545 | CAMK2N1 | 1.946609706 | 0.000825 | 0.148774 |
| ENSG00000168528 | SERINC2 | 1.462363964 | 0.000841 | 0.148774 |
| ENSG00000186222 | CNO | 1.76452142 | 0.000843 | 0.148774 |
| ENSG00000101489 | CELF4 | 1.251710434 | 0.000985 | 0.170642 |
| ENSG00000176428 | VPS37D | 1.630001754 | 0.001063 | 0.176316 |
| ENSG00000148362 | C9orf142 | 1.603570884 | 0.001082 | 0.176316 |
| ENSG00000059728 | MXD1 | 1.473292561 | 0.001091 | 0.176316 |
| ENSG00000188322 | SBK1 | 1.423210412 | 0.001111 | 0.176316 |
| ENSG00000197696 | NMB | 1.559897931 | 0.001161 | 0.176316 |
| ENSG00000232112 | CCDC72 | 1.532010783 | 0.001173 | 0.176316 |
| ENSG00000130479 | MAP1S | 1.074107659 | 0.001181 | 0.176316 |
| ENSG00000160949 | TONSL | 1.252459173 | 0.001186 | 0.176316 |
| ENSG00000069535 | MAOB | −1.324401149 | 0.001187 | 0.176316 |
| ENSG00000156026 | MCU | 1.214553548 | 0.001267 | 0.17637 |
| ENSG00000121410 | A1BG | 1.571070495 | 0.001297 | 0.17637 |
| ENSG00000185813 | PCYT2 | 1.754396066 | 0.001302 | 0.17637 |
| ENSG00000155130 | MARCKS | 1.184759392 | 0.001304 | 0.17637 |
| ENSG00000184990 | SIVA1 | 1.619959322 | 0.001338 | 0.17637 |
| ENSG00000228716 | DHFR | −1.348258672 | 0.001343 | 0.17637 |
| ENSG00000162073 | PAQR4 | 1.469743494 | 0.001349 | 0.17637 |
| ENSG00000185262 | FAM100B | 1.098596912 | 0.001352 | 0.17637 |
| ENSG00000177045 | SIX5 | 1.309255712 | 0.001366 | 0.17637 |
| ENSG00000162066 | AMDHD2 | 1.569572369 | 0.001376 | 0.17637 |
| ENSG00000034693 | PEX3 | −1.540503965 | 0.001443 | 0.18032 |
| ENSG00000100968 | NFATC4 | 1.561779065 | 0.001445 | 0.18032 |
| ENSG00000143013 | LMO4 | 1.694528514 | 0.001471 | 0.181124 |
| ENSG00000160570 | DEDD2 | 1.636128138 | 0.001523 | 0.182876 |
| ENSG00000160932 | LY6E | 1.360126717 | 0.001524 | 0.182876 |
| ENSG00000205323 | SARNP | 1.440943784 | 0.001582 | 0.187401 |
| ENSG00000103855 | CD276 | 1.272258918 | 0.001631 | 0.190668 |
| ENSG00000144485 | HES6 | 2.006311401 | 0.00165 | 0.190668 |
| ENSG00000185129 | PURA | 1.54426728 | 0.001751 | 0.199833 |
| ENSG00000114646 | CSPG5 | 1.545083925 | 0.001839 | 0.20735 |
| ENSG00000253276 | CCDC71L | 1.472234479 | 0.00191 | 0.212771 |
| ENSG00000148730 | EIF4EBP2 | 1.451786364 | 0.001942 | 0.213789 |
| ENSG00000149357 | LAMTOR1 | 1.581067574 | 0.002003 | 0.218007 |
| ENSG00000130731 | C16orf13 | 1.307690059 | 0.002029 | 0.218251 |
| ENSG00000161677 | JOSD2 | 1.327521422 | 0.002062 | 0.219267 |
| ENSG00000108963 | DPH1 | 1.298439642 | 0.00214 | 0.225026 |
| ENSG00000171282 | BAHCC1 | 1.257616515 | 0.002241 | 0.229915 |
| ENSG00000136274 | NACAD | 0.948987658 | 0.00226 | 0.229915 |
| ENSG00000108387 | SEPT10 | 1.627880773 | 0.002263 | 0.229915 |
| ENSG00000173614 | NMNAT1 | −1.588025343 | 0.002285 | 0.229915 |
| ENSG00000166166 | TRMT61A | 1.339327969 | 0.002321 | 0.231067 |
| ENSG00000082458 | DLG3 | 1.358929899 | 0.002404 | 0.235325 |
| ENSG00000130589 | RP4-697K14.7.1 | 1.330689333 | 0.002414 | 0.235325 |
| ENSG00000157833 | FAM59B | 2.050070243 | 0.002467 | 0.238064 |
| ENSG00000147536 | GINS4 | −1.301777547 | 0.002506 | 0.238906 |
| ENSG00000116649 | SRM | 1.40200708 | 0.002527 | 0.238906 |
| ENSG00000240972 | MIF | 1.021447804 | 0.00261 | 0.242052 |
| ENSG00000182809 | CRIP2 | 1.201456759 | 0.002612 | 0.242052 |
| ENSG00000183780 | SLC35F3 | 1.516811748 | 0.002653 | 0.242114 |
| ENSG00000141570 | CBX8 | 1.237642849 | 0.002665 | 0.242114 |
| ENSG00000158526 | TSR2 | −1.174094346 | 0.002747 | 0.247217 |
| ENSG00000159596 | TMEM69 | −1.334161523 | 0.002838 | 0.252933 |

Known translation targets of FMRP have been reported to include eEF2, eEF1, all three eIF4G isoforms, TSC2 and SYNGAP1. Consistent with these reports, the sequencing data showed that for the knockdown of FMRP, the elongation factors (eEF2 and eEF1), as well as TSC2 and SYNGAP1, had an associated increase in translational rate (increased translation of these targets) by 30-50% in the absence of changes of RNA level. In contrast, no changes in either RNA level or translational rate was observed for the three eIF4G isoforms.

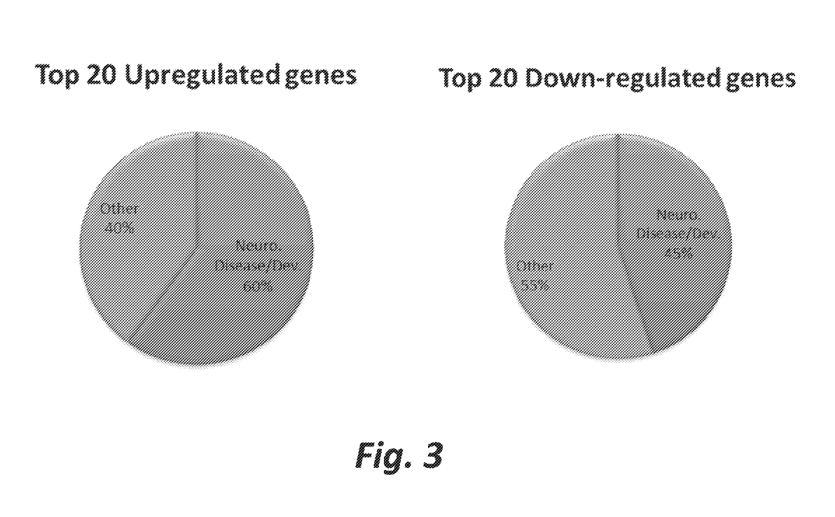

The set of genes identified via changes in translational efficiency or translational rate upon knockdown of the FMR1 gene was quite distinct from the corresponding set based on transcription. Of particular interest were the top 20 up- or down-regulated genes (log₂ fold increase of 1.9-3.5 (p-value≦0.001) or decrease of 1.5-2.2 (p-value≦0.05), respectively) from changes in translational efficiency (see Tables 2 and 3). Of these 40 genes, only three also had significant (p<0.05) movement in mRNA levels. As shown in FIG. 3, 60 and 45% of these 20 translationally up- and 20 down-regulated genes, respectively, are associated with a neurological disorder (neurodegenerative, neurodevelopmental, neurocognitive). This enrichment for neurological association increased to 70% and 50% for the top 10 up- and down-regulated genes, respectively.

TABLE 2
Top Upregulated Genes

| | TE Log₂ | |
|---|---|---|
| Gene* | Ratio | Gene Description |
| CTXN1 | 3.473 | cortexin 1 |
| | | [Source: HGNC Symbol; Acc: 31108] |
| IRGQ | 2.769 | immunity-related GTPase family, Q |
| | | [Source: HGNC Symbol; Acc: 24868] |
| UBE2D4 | 2.398 | ubiquitin-conjugating enzyme E2D 4 |
| | | (putative) |
| | | [Source: HGNC Symbol; Acc: 21647] |
| MAPK11 | 2.373 | mitogen-activated protein kinase 11 |
| | | [Source: HGNC Symbol; Acc: 6873] |
| CHMP3 | 2.222 | charged multivesicular body protein 3 |
| | | [Source: HGNC Symbol; Acc: 29865] |
| MYD88 | 2.167 | myeloid differentiation primary response 88 |
| | | [Source: HGNC Symbol; Acc: 7562] |
| BLOC1S2 | 2.155 | biogenesis of lysosomal organelles |
| | | complex-1, subunit 2 |
| | | [Source: HGNC Symbol; Acc: 20984] |
| GDF11 | 2.141 | growth differentiation factor 11 |
| | | [Source: HGNC Symbol; Acc: 4216] |
| BCL7B | 2.055 | B-cell CLL/lymphoma 7B |
| | | [Source: HGNC Symbol; Acc: 1005] |
| HES6 | 2.006 | hairy and enhancer of split 6 (Drosophila) |
| | | [Source: HGNC Symbol; Acc: 18254] |
| ZNF358 | 2.108 | zinc finger protein 358 |
| | | [Source: HGNC Symbol; Acc: 16838] |
| FAM59B | 2.050 | GRB2 associated, regulator of MAPK1-like |
| | | [Source: HGNC Symbol; Acc: 27172] |
| CISD3 | 2.022 | CDGSH iron sulfur domain 3 |
| | | [Source: HGNC Symbol; Acc: 27578] |
| SLC39A4 | 1.981 | solute carrier family 39 (zinc transporter), |
| | | member 4 |
| | | [Source: HGNC Symbol; Acc: 17129] |
| CAMK2N1 | 1.947 | calcium/calmodulin-dependent protein |
| | | kinase II inhibitor 1 |
| | | [Source: HGNC Symbol; Acc: 24190] |
| ZNF579 | 1.930 | zinc finger protein 579 |
| | | [Source: HGNC Symbol; Acc: 26646] |
| C1QTNF1 | 1.925 | C1q and tumor necrosis factor related protein 1 |
| | | [Source: HGNC Symbol; Acc: 14324] |
| PPP1R3F | 1.895 | protein phosphatase 1, regulatory subunit 3F |
| | | [Source: HGNC Symbol; Acc: 14944] |
| CHCHD5 | 1.895 | coiled-coil-helix-coiled-coil-helix |
| | | domain containing 5 |
| | | [Source: HGNC Symbol; Acc: 17840] |
| DMD | 1.882 | dystrophin |
| | | [Source: HGNC Symbol; Acc: 2928] |
| YDJC | 1.878 | YdjC homolog (bacterial) |
| | | [Source: HGNC Symbol; Acc: 27158] |

*Genes highlighted in bold have an association with a neurological disorder.

TABLE 3
Top Downregulated Genes

| | TE Log₂ | |
|---|---|---|
| Gene* | Ratio | Gene Description |
| CIB1 | −2.157 | calcium and integrin binding 1 (calmyrin) |
| | | [Source: HGNC Symbol; Acc: 16920] |
| SNPH | −1.917 | syntaphilin |
| | | [Source: HGNC Symbol; Acc: 15931] |
| MAPK6 | −1.909 | mitogen-activated protein kinase 6 |
| | | [Source: HGNC Symbol; Acc: 6879] |
| CMC2 | −1.904 | COX assembly mitochondrial protein |
| | | 2 homolog (S. cerevisiae) |
| | | [Source: HGNC Symbol; Acc: 24447] |
| ZDHHC21 | −1.883 | zinc finger, DHHC-type containing 21 |
| | | [Source: HGNC Symbol; Acc: 20750] |
| TXNL4B | −1.815 | thioredoxin-like 4B |
| | | [Source: HGNC Symbol; Acc: 26041] |
| GCLM | −1.731 | glutamate-cysteine ligase, modifier subunit |
| | | [Source: HGNC Symbol; Acc: 4312] |
| EVI5 | −1.705 | ecotropic viral integration site 5 |
| | | [Source: HGNC Symbol; Acc: 3501] |
| MTSS1 | −1.705 | metastasis suppressor 1 |
| | | [Source: HGNC Symbol; Acc: 20443] |
| BAG2 | −1.702 | BCL2-associated athanogene 2 |
| | | [Source: HGNC Symbol; Acc: 938] |
| FAM200A | −1.620 | family with sequence similarity 200, member A |
| | | [Source: HGNC Symbol; Acc: 25401] |
| GORAB | −1.606 | golgin, RAB6-interacting |
| | | [Source: HGNC Symbol; Acc: 25676] |
| NMNAT1 | −1.588 | nicotinamide nucleotide adenylyltransferase 1 |
| | | [Source: HGNC Symbol; Acc: 17877] |
| NOMO2 | −1.542 | NODAL modulator 2 |
| | | [Source: HGNC Symbol; Acc: 22652] |
| PEX3 | −1.541 | peroxisomal biogenesis factor 3 |
| | | [Source: HGNC Symbol; Acc: 8858] |
| C21orf91 | −1.513 | chromosome 21 open reading frame 91 |
| | | [Source: HGNC Symbol; Acc: 16459] |
| TMEM19 | −1.506 | transmembrane protein 19 |
| | | [Source: HGNC Symbol; Acc: 25605] |
| ZNF642 | −1.491 | ZFP69 zinc finger protein |
| | | [Source: HGNC Symbol; Acc: 24708] |
| CCDC41 | −1.482 | coiled-coil domain containing 41 |
| | | [Source: HGNC Symbol; Acc: 17966] |
| SLC16A10 | −1.479 | solute carrier family 16 (aromatic |
| | | amino acid transporter), member 10 |
| | | [Source: HGNC Symbol; Acc: 17027] |
| ZNF596 | −1.476 | zinc finger protein 596 |
| | | [Source: HGNC Symbol; Acc: 27268] |

*Genes highlighted in bold have an association with a neurological disorder.

### Conclusion

Fragile X is the most inheritable form of mental retardation. Current concepts of how FMRP regulates the translation of specific mRNAs are still being elucidated. This example shows that ribosome profiling and pathway analysis of genome-wide translational efficiencies after FMRP knockdown translationally regulates genes that are highly associated with various neurological disorders providing a novel insight into the key genes that are translationally regulated. The genes identified represent a new set of validated targets for points of intervention for the treatment of, for example, fragile X syndrome.

The various embodiments described above can be combined to provide further embodiments. All of the U.S. patents, U.S. patent application publications, U.S. patent applications, foreign patents, foreign patent applications and non-patent publications referred to in this specification, including but not limited to U.S. Patent Application No. 61/937,315, are incorporated herein by reference in their entirety.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for preventing, treating or ameliorating a neurological disorder, comprising administering to a subject having a neurological disorder a therapeutically effective amount of a modulator of any one or more of the genes listed in Table 2 or Table 3, or any encoded products thereof.

2. A method for reducing the risk of developing a neurological disorder, comprising: administering to a subject at risk of developing a neurological disorder a therapeutically effective amount of a modulator of any one or more of the genes listed in Table 2 or Table 3, or any encoded products thereof.

3. The method according to claim 1, wherein the modulator is formulated with a pharmaceutically acceptable excipient.

4. The method according to claim 1, wherein the modulator is administered in combination with a second therapeutic agent.

5. The method according to claim 1, wherein the neurological disorder is selected from Parkinson's disease, Amyotrophic Lateral Sclerosis (ALS), Creutzfeldt-Jakob disease, Huntington's disease, Lewy body dementia, frontotemporal dementia, corticobasal degeneration, primary progressive aphasia, progressive supranuclear palsy or Alzheimer's disease.

6. The method according to claim 1, wherein the neurological disorder is selected from autism, autism spectrum disorders, Fragile X Syndrome, attention deficit disorder, or pervasive development disorders.

7. The method according to claim 1, wherein the subject is a human.

8. A method for identifying a candidate therapeutic for normalizing a translational profile associated with a neurological disorder, comprising:
(a) determining three independent translational profiles, each for a plurality of genes, wherein (i) a first translational profile is from a neurological disorder sample, (ii) a second translational profile is from (1) a control non-diseased sample or (2) a control non-diseased sample contacted with a candidate agent, and (iii) a third translational profile is from the neurological disorder sample contacted with a candidate agent;
(b) determining a first differential translational profile comprising one or more genes differentially translated in the first translational profile as compared to the second translational profile, and determining a second differential translational profile comprising one or more genes differentially translated in the first translational profile as compared to the third translational profile, wherein the one or more differentially translated genes are selected from the genes listed in Table 2 or Table 3; and
(c) identifying the agent as a candidate therapeutic for normalizing a translational profile associated with the neurological disorder when the first differential translational profile is comparable to the second differential translational profile.

9. The method according to claim 8, wherein the neurological disorder is selected from Parkinson's disease, Amyotrophic Lateral Sclerosis (ALS), Creutzfeldt-Jakob disease, Huntington's disease, Lewy body dementia, frontotemporal dementia, corticobasal degeneration, primary progressive aphasia, progressive supranuclear palsy or Alzheimer's disease.

10. The method according to claim 8, wherein the neurological disorder is selected from autism, autism spectrum disorders, Fragile X Syndrome, attention deficit disorder, or pervasive development disorders.

11. A method for validating a target for normalizing a translational profile associated with a neurological disorder, the method comprising:
(a) determining three independent translational profiles, each for a plurality of genes, wherein (i) a first translational profile is from a neurological disorder, a neurodegenerative disease, a neurodevelopmental disease, a metabolic disease, or a viral infection sample, (ii) a second translational profile is from (1) a control non-diseased sample or (2) a control non-diseased sample contacted with an agent that modulates a target, and (iii) a third translational profile is from the neurological disorder sample contacted with the agent that modulates the target;
(b) determining a first differential translational profile comprising one or more genes differentially translated in the first translational profile as compared to the second translational profile, and determining a second differential translational profile comprising one or more genes differentially translated in the first translational profile as compared to the third translational profile, wherein the one or more differentially translated genes are selected from the genes listed in Table 2 or Table 3; and
(c) validating the target as a target for normalizing a translational profile associated with the neurological disorder when the first differential translational profile is comparable to the second differential translational profile.

12. The method according to claim 11, wherein the neurological disorder is selected from Parkinson's disease, Amyotrophic Lateral Sclerosis (ALS), Creutzfeldt-Jakob disease, Huntington's disease, Lewy body dementia, frontotemporal dementia, corticobasal degeneration, primary progressive aphasia, progressive supranuclear palsy or Alzheimer's disease.

13. The method according to claim 11, wherein the neurological disorder is selected from autism, autism spectrum disorders, Fragile X Syndrome, attention deficit disorder, or pervasive development disorders.

14. A method of identifying a subject as a candidate for treating a neurological disorder with a therapeutic agent, the method comprising:
(a) determining a first translational profile for a plurality of genes in a sample from a subject having or suspected of having a neurological disorder;
(b) determining a second translational profile for a plurality of genes in a control sample, wherein the control sample is from a subject known to respond to the therapeutic agent and wherein the sample has not been contacted with the therapeutic agent; and
(c) identifying the subject as a candidate for treating neurological disorder with the therapeutic agent when the translational profile for one or more genes selected from Table 2 or Table 3 of the first translational profile are comparable to the translational profile of the corresponding genes in the second translational profile.

15. A method for treating a neurological disorder, comprising administering a therapeutic agent to a subject identified according to the method of claim 12, thereby treating the subject.

16. The method according to claim 14, wherein the neurological disorder is selected from Parkinson's disease, Amyotrophic Lateral Sclerosis (ALS), Creutzfeldt-Jakob disease, Huntington's disease, Lewy body dementia, frontotemporal dementia, corticobasal degeneration, primary progressive aphasia, progressive supranuclear palsy or Alzheimer's disease.

17. The method according to claim 14, wherein the neurological disorder is selected from autism, autism spectrum disorders, Fragile X Syndrome, attention deficit disorder, or pervasive development disorders.
